(19) United States
(12) Patent Application Publication
(10) Pub. No.: US 2010/0272639 A1
(43) Pub. Date: Oct. 28, 2010

(54) POLYSACCHARIDE NANOPARTICLES

(76) Inventor: John Robert Dutcher, Guelph (CA)

Correspondence Address: Lorelei G. Graham;c/o Macleod Dixon LLP, Toronto-Dominion Centre, TD Waterhouse Tower, 79 Wellington Street West, Suite 2300, PO Box 128, Toronto, ON, M5K 1H1, CA

(21) Appl. No.: 12/809,629

(22) PCT Filed: Dec. 19, 2008
(86) PCT No.: PCT/IB08/03958

§ 371 Date: Jun. 21, 2010

Related U.S. Application Data
(60) Provisional application No. 61/016,418, filed on Dec. 21, 2007.

(51) Int. Cl. A61K 9/14; C07H 3/06; C07K 16/00; A61K 39/00; C07K 14/555; C07H 21/02; C07H 21/00; A61K 31/045; A61K 31/095; A61K 31/275; A61K 31/4164; A61K 31/19; A61K 49/00; A61K 51/06; A61K 8/02; A61Q 90/00; A61P 31/00; A61P 37/00; A61P 25/00; A61P 25/16; A61P 33/00; A61P 29/00; A61P 23/02; A61P 3/10; C12N 9/98; C12P 19/00; C07H 1/00

(52) U.S. Cl. 424/1.37; 536/123.1; 530/391.1; 424/185.1; 530/351; 536/23.1; 536/24.5; 424/499; 514/724; 514/706; 514/519; 514/403; 514/557; 424/9.1; 424/401; 435/187; 435/72; 428/402

(57) ABSTRACT

Polysaccharide nanoparticles that are particularly useful in for example drug and agent delivery, tissue-specific targeting, for medical imaging and diagnosis, as well as modifiers of physico-chemical properties. The nanoparticles can be highly-branched glucose homopolymers and can be characterized by a uniform spherical shape. They are monodisperse, hydrophilic and produce low solution viscosities. The nanoparticles are non-toxic, biocompatible and biodegradable. Also, the process of isolation of said polysaccharide nanoparticles from various organisms including, but not limited to, microorganisms such as bacteria and yeasts. Also provided are methods for chemical conjugation of the polysaccharide nanoparticles with various agents. Also provided are examples of use of the polysaccharide nanoparticles and their derivatives as drug delivery systems and fluorescent di-agnostics.

### CROSS-REFERENCE TO RELATED APPLICATIONS

This application claims priority to U.S. provisional application Ser. No. 61/016,418, filed Dec. 21, 2007 to Dutcher et al., which is hereby incorporated by reference in its entirety.

### BACKGROUND

Nanoparticles are being extensively investigated for their benefits in biomedical applications such as, for example, therapeutic agents and gene delivery, medical imaging, diagnosis, and tissue targeting. However, for medical applications and especially human health care, there can be stringent material requirements. Some of the more important requirements include, for example, low toxicity and biocompatibility of the material. Furthermore, it is very desirable for medical applications that nanoparticles be biodegradable, hydrophilic, and non-immunogenic. Monodispersity is another very desirable feature of nanoparticles, since size may greatly influence the distribution and accumulation of the nanoparticles in biological tissues, as well as pharmokinetics. Furthermore, nanoparticle surface modification and derivatization occurs much more predictably if the nanoparticles are monodisperse. However, limited progress has been achieved in identifying suitable nanomaterials with combination of these and other desirable properties. To date, there are only a few examples of polymer-based nanoparticles which are monodisperse and have all of the favorable properties such as non-toxicity, non-immunogenicity and biodegradability. Common inorganic nanoparticles, such as quantum dots, carbon nanotubes, fullerenes may have serious issues with respect to toxicity and biocompatibility. Various polymer based nanoparticles, both synthetic and naturally extracted, have been researched, but it is technically difficult to produce polymers in monodisperse form. The most prominent example of monodisperse polymeric nanoparticles is that of highly branched molecules called dendrimers, with peptide and polysaccharide-based dendrimers being most suitable for biomedical applications. However, the cost of such dendrimers, especially dendrimers of high molecular weight, presently is prohibitively high due to technical difficulties in their synthesis.

### SUMMARY

Various embodiments described herein include compositions, individual particles and nanoparticles and collections of particles and nanoparticles, methods of making and methods of using compositions, and further formulations and devices.

For example, one embodiment provides a composition comprising nanoparticles comprising branched polysaccharide and wherein the nanoparticles are substantially monodisperse in size.

Another embodiment is a composition comprising nanoparticles purified from a source, wherein the nanoparticles comprise at least one branched polysaccharide, and the nanoparticles are substantially spherical and substantially monodisperse in size.

Another embodiment is a composition comprising optionally functionalized nanoparticles comprising branched polysaccharide and wherein the nanoparticles are substantially monodisperse in size.

Other embodiments provide for a composition comprising nanoparticles comprising branched polysaccharide and wherein the nanoparticles are substantially monodisperse in size; a method of producing a polysaccharide nanoparticle; a method of derivatizing the polysaccharide nanoparticles; a method of using a composition for drug delivery; a method of using a composition for diagnosis of a disease or medical condition, a method of using the nanoparticle for blood substitute product; and a method of using a composition for cosmetic formulation.

Provided herein are methods for producing, isolating and functionalizing monodisperse polysaccharide nanoparticles (nanoPS) which are non-toxic, biocompatible and biodegradable. Furthermore, the nanoPS can be composed of a high molecular weight glucose homopolymer that is structurally similar to glycogen.

NanoPS molecules can be hydrophilic, highly soluble in water and produce low solution viscosities. They can be functionalized and derivatized using common carbohydrate chemistry. nanoPS can be produced with purities that meet the stringent requirements for biomedical polymers, e.g. for enteral and especially for parenteral administration of drugs. Production of nanoPS can be scaled up using fermentation and purification techniques that have been well developed in the biotechnological sector which will produce a low cost product that can be used for applications usually targeted by dendrimer chemistry.

In one aspect, provided herein are monodisperse polysaccharide nanoparticles (nanoPS) that are useful in drug and agent delivery, for medical imaging, molecular diagnostics and molecular targeting, as well as modifiers of physico-chemical properties. nanoPS molecules comprising α-D-glucose chains with 1→4 linkage and branching points occurring at 1→6 and with a degree of branching having the range of about 6 to about 13%, with a structure that is similar to that reported for glycogen contained in animal tissue. nanoPS molecules have a spherical shape as determined using dynamic light scattering. The nanoPS molecules are very monodisperse in molecular weight, with polydispersity index (M_{W}/Mₙ) values that vary between about 1.000 and about 1.100, depending on the source and purification and isolation method. The corresponding weight average molecular weight (M_{w}) ranges from about 2.00×10⁶ to about 25.00×10⁶ daltons, as determined using size exclusion chromatography (SEC). Depending on the source and purification and isolation method, the nanoPS molecule diameter can be varied from, for example, about 20 to about 60 nm, or in other embodiments, from about 20 nm to about 350 nm, as determined using multi-angle laser light scattering (MALLS) and atomic force microscopy (AFM). nanoPS is highly soluble in aqueous solutions and aprotic polar organic solvents. The combination in some embodiments of molecule size in the range of tens of nanometers, high molecular weight, monodispersity and high solubility can make nanoPS suitable for a wide range of industrial and biomedical applications.

In another aspect, provided herein are methods for producing nanoPS which comprise (a) cultivation of microorganisms in appropriate media, followed by (b) isolation of nanoPS according to the procedures described herein.

In yet another aspect, provided herein are various functional products prepared by chemical conjugation of nanoPS molecules with various active compounds and use thereof in various applications, such as drug delivery systems, MRI/CT contrast agents, fluorescent diagnostics, blood substitute products, and applications in foods and cosmetic formulations.

One or more advantages of at least some of the embodiments described herein include: (i) particles of nanoPS are non-toxic, biocompatible and biodegradable and suitable for parenteral administration, e.g., by injection or by infusion, either transmucosal or inhalational; (ii) nanoPS can be produced at a significantly lower cost compared to synthetic polysaccharide-based dendrimers; and/or (iii) a broad variety of microorganisms can be used for the production of nanoPS, such as bacteria, yeasts, microalgae and cyanobacteria. In particular, the nanoparticles can be highly soluble or dispersible and can be engineered with well-controlled properties, similar to synthetic polymers.

### BRIEF DESCRIPTION OF THE DRAWINGS

FIGS. 1A and 1B show (A) a size exclusion chromatography (SEC) plot and (B) an atomic force microscopy (AFM) image obtained for nanoPS prepared accordingly to Example 3. The SEC plot in (A) comprises a single, narrow peak. The inset in (A) lists parameter values for the nanoPS molecules. The inset in (B) shows the Fast Fourier Transform of the AFM image which demonstrates the dense ordered packing of the nanoPS molecules because of their high monodispersity.

FIGS. 2A and 2B show (A) a size exclusion chromatography (SEC) plot and (B) an atomic force microscopy (AFM) image obtained for nanoPS prepared accordingly to Example 2. The SEC plot in (A) comprises a single, narrow peak. The inset in (A) lists parameter values for the nanoPS molecules.

FIGS. 3A and 3B show (A) a size exclusion chromatography (SEC) plot and (B) an atomic force microscopy (AFM) image obtained for nanoPS prepared using method accordingly to Example 5. The SEC plot in (A) comprises of a single, narrow peak. The inset in (A) lists parameter values for the nanoPS molecules.

FIG. 4. GC-MS spectrum of permethylated alditol acetates obtained for nanoPS isolated in Example 3.

FIG. 5. ¹H NMR spectrum obtained at 42° C. for nanoPS isolated in Example 3.

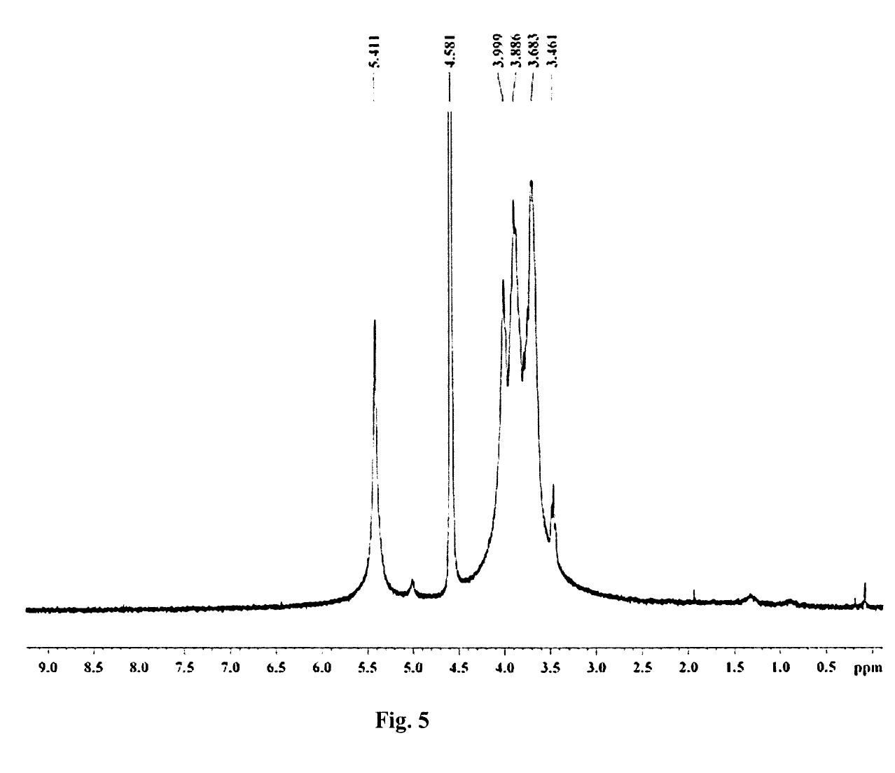

FIG. 6. Dynamic Light Scattering plot of the polysaccharide nanoparticles prepared in accordance with Example 3 of the present invention.

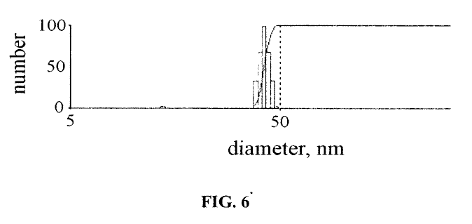

FIG. 7. shows a fluorescence microscopy image of polysaccharide nanoparticle-Rhodamine B conjugates from Example 16 (orange fluorescence) taken up by normal murine endothelial cell lines after 16 hrs incubation. The polysaccharide nanoparticles were accumulated only in the cytoplasm. N: nucleus.

### DETAILED DESCRIPTION

Priority U.S. provisional application Ser. No. 61/016,418, filed Dec. 21, 2007 to Dutcher et al. is hereby incorporated by reference in its entirety including the claims, working examples, figures, and other subsections of the application. Seven other references are cited below and cited to throughout this application. All references cited herein are hereby incorporated by reference in their entireties.

Various embodiments described herein relate to polysaccharide nanoparticles (nanoPS) that are useful in drug and agent delivery, tissue-specific targeting, for medical imaging and diagnosis, in cosmetic formulations, functional foods, as well as modifiers of physico-chemical properties.

Synthesis and characterization of polymers and particles thereof are generally known in the art. See for example Billmeyer, Textbook of Polymer Science, 3^{rd} Ed, Wiley, 1984; Allcock et al., Contemporary Polymer Chemistry, Prentice-Hall, 1981.

### Polymer Material Characterization

Polysaccharides and carbohydrates are widely presented in nature and are generally known in the art. See, for example, Bohinski, Modern Concepts in Biochemistry, 4^{th} Ed., Allyn and Bacon, 1983; Allcock et al., Contemporary Polymer Chemistry, Prentice-Hall, 1981. Polysaccharides can comprise single monomer species (homopolymers) or multiple monomer species (heteropolymers), and can be linear or branched (see, for example, Bohinski (1983) and Allcock (1981)). Branched polysaccharide homopolymers of glucose species are generally known in the art (see, for example, Alberts et al., Molecular Biology of the Cell, 4^{rd} Ed., Garland Publishing, 2002). The most prominent examples are glycogen in animals and amylopectin in plants which both have energy storage functions. Both glycogen and amylopectin comprise glucose units which are linked by α-1,4 glycosidic bonds, and the branching created through α-1,6 glycosidic bond with a second glucose unit. The degree of branching (DB) is given by the ratio of the number of glucose units which have branching points (α-1,6 linkages) to the total number of glucose units and can be expressed in mol %. It is generally assumed that amylopectin has lower DB values (3-7 mol %) than glycogen (7-15 mol %), but the values depend on the origin and preparation of the sample and the experimental method used and therefore differentiation between amylopectin and glycogen based on the DB values is elusive. For example, the DB of nanoPS can be within the range of about 6 to about 13 mol %.

The molecular weight of a polymer can be characterized by the weight average molecular weight (M_{w}) and the number average molecular weight (Mₙ), and can be measured by methods known in the art including, for example, light scattering and size exclusion chromatography. For example, the M_{w} value of nanoPS can be within the range of about 1×10⁶ to about 25×10⁶, or about 2×10⁶ to about 25×10⁶.

The distribution of the molecular weight of polymer molecules is characterized by the polydispersity index (PDI) which is defined as the ratio of M_{w} to Mₙ. For example, nanoPS can have PDI values which range from about 1.000 to about 1.300, or about 1.000 to about 1.100.

The polysaccharide nanoparticles can comprise or consist essentially of other components within the particle beyond the glucose polymer to the extent the basic and novel features described herein are not substantially compromised.

### Nanoparticle Characterization

Nanoparticles are generally known in the art. See for example Poole et al., Introduction to Nanotechnology, Wiley, 2003; Nanobiotechnology II (Eds. Mirkin and Niemeyer), Wiley-VCH, 2007.

Nanoparticle size, including distributions (dispersity) and average values of the diameter, can be measured by methods known in the art. These primarily include microscopy techniques, e.g. transmission electron microscopy and atomic force microscopy. For example, the average diameter of nanoPS can be about 20 nm to about 60 nm, or in other embodiments, from about 20 nm to about 350 nm.

It is generally known in the art that nanoparticle systems can be characterized by low size polydispersity, i.e. monodispersity. See for example Nanoparticles: From Theory to Application (Ed. Schmid), Wiley-VCH, 2006. The size polydispersity can be described in % by the width of the size distribution histogram measured at the 50% of the peak height divided by mean nanoparticle size and multiplied by 100%. For example, the size polydispersity of nanoPS can be from about 4% to about 50%.

NanoPS can be used in dispersions and other formulations with use of solvent and dispersant systems including aqueous, non-aqueous, and mixed aqueous-nonaqueous systems. Organic solvents can include for example polar aprotic solvents, e.g., dimethyl sulfoxide (DMSO), and dimethyl formamide (DMF). The pH of the solvent can be for example about 3.0-11.0. The concentration of solids in the solution can be for example up to 30% (by mass) with no detectable nanoparticles aggregation or precipitation. nanoPS solutions have no detectable light absorption in the UV and visible range of wavelengths. Aqueous solutions of nanoPS have low viscosity at relatively high concentrations of up to 30% (by mass).

NanoPS molecules assemble into densely packed, ordered films on various flat surfaces. The surface of nanoPS molecules can contain several thousands of terminal hydroxyl functional groups, which can be further modified with other functional groups. nanoPS molecules are generally neutral over a wide range of pH.

### Preparation and Isolation of NanoPS

Various embodiments described herein relate to the cultivation of microorganisms under appropriate conditions with a subsequent isolation of nanoPS particles from bacterial biomass. The nanoparticles can be purified from sources such as biomass including bacterial biomass.

The use of bacteria is preferable since the process can be performed in batch mode or by using continuous fermentation. This is a scalable and consistent process, which can be conducted in such a way that it yields biomass which does not have other large molecular weight polysaccharides such as amylopectin and amylose, and is free of pathogenic bacteria, parasites, viruses and prions associated with shellfish or animal tissues.

In one embodiment, Gram-negative bacteria are used, which lack thick, rigid cell walls, making the initial step of cell disintegration (before extraction procedure) easier or unnecessary.

In one embodiment, rough strains of Gram-negative bacteria are used, which produce no capsular material and which express only rough lipopolysaccharide (LPS), i.e., LPS molecules which lack high molecular weight O-side chains and are terminated only with a core oligosaccharide. The use of rough strains will decrease the amount of other high-molecular weight polysaccharides in microbial cells and, therefore, greatly facilitate the separation and purification of nanoPS molecules.

In one embodiment, rough strains of Escherichia coli, e.g., E. coli K12 are used, since these strains have many advantageous characteristics, such as fast growth using inexpensive media, they are accepted for use in the pharmaceutical industry, and the background for large-scale fermentation of these strains is well established. Furthermore, the genome of this bacterium is completely sequenced and genetic engineering alterations/manipulations can be performed by those experienced in the art to generate strains which have a high yield of nanoPS.

The amount of nanoPS synthesized by microorganisms depends on the cultivation conditions such as temperature, pH, dissolved oxygen concentration, growth medium composition, etc. In some instances, the production of nanoPS is significantly increased when the growth of the microorganisms is limited by the absence of certain minerals, such as phosphorus, sulfur, and especially nitrogen, or limited by growth factors, e.g., essential amino acids.

In one embodiment, E. coli K12 is cultivated using a two stage procedure. In some instances, the first fermentation is performed in a growth medium containing all of the necessary mineral elements, and then the bacterial cells are transferred into the same growth medium with the exception that the nitrogen source is excluded from the medium composition. Growth in such conditions, with an excess carbon source but limited by nitrogen, results in a high yield of nanoPS.

One embodiment uses a genetically modified strain of E. coli for cultivation according to the previous embodiment with the aim of obtaining higher yields of nanoPS.

In another embodiment, a rough strain of Geobacter sulfurreducens is used for nanoPS molecule production. G. sulfurreducens is a Gram-negative, strictly anaerobic bacterium which is capable of anaerobic respiration of fumarate. The medium composition provides an excess of the carbon source, sodium acetate, which also serves as an electron donor. However upon bacterial fermentation, an electron acceptor, sodium fumarate, becomes depleted and, therefore, limits the growth. This results in a significant increase in nanoPS accumulation in bacterial cells.

After completion of the cultivation process, bacterial cells are separated from the growth medium by centrifugation or by other means e.g., by ultrafiltration. This produces wet, concentrated biomass.

Another aspect provides a process for the isolation of nanoPS from bacterial biomass. Although this can be achieved in different ways, variants of the process typically use the following steps:
1. Cell disintegration by French pressing, or by chemical treatment, e.g., with phenol;
2. Separation of insoluble cell components, e.g., cell walls, by centrifugation;
3. Elimination of proteins and nucleic acids from cell lyzate by enzymatic treatment followed by dialysis which produces an extract containing crude polysaccharides and LPS;
4. Elimination of LPS by weak acid hydrolysis, or by treatment with salts of multivalent cations such as Mg²⁺, Al³⁺, etc., preferably Ca²⁺, which results in the precipitation of insoluble LPS products;
5. Purification of the nanoPS enriched fraction by dialysis and/or size exclusion chromatography;
6. Precipitation of nanoPS with a suitable organic solvent such as acetone, methanol, propanol, etc., preferably ethanol. Alternatively, a concentrated nanoPS solution can be obtained by ultrafiltration or by ultracentrifugation;
7. Freeze drying to produce a powder of nanoPS.

Other types of sources can be used as known in the art. See, for example, Smith, Biotechnology, 4^{th} Ed., Cambridge University Press, 2004. These methods of polysaccharide nanoparticle isolation can be applied to biological material other than that derived from microorganisms. For example, in some embodiments, polysaccharide nanoparticles can be isolated from animals or plants including for example oysters and rice.

Chemical Functionalization of nanoPS

The present embodiments also provide nanoparticles and molecules with chemically functionalized surface and/or nanoparticles conjugated with a wide array of molecules. Chemical functionalization is known in the art of synthesis. See, for example, March, Advanced Organic Chemistry, 6^{th} Ed., Wiley, 2007. Functionalization can be carried out on the surface of the particle, or on both the surface and the interior of the particle.

Such functionalized surface groups include, but are not limited to, nucleophilic and electrophilic groups, acidic and basic groups, including for example carbonyl groups, amine groups, thiol groups, carboxylic or other acidic groups. Amino groups can be primary, secondary, tertiary, or quaternary amino groups. nanoPS also can be functionalized with unsaturated groups such as vinyl and allyl groups.

The nanoparticles, as isolated and purified, can be either directly functionalized or indirectly one or more intermediate linkers or spacers can be used. The nanoparticles can be subjected to one or more than one functionalization steps including two or more, three or more, or four or more functionalization steps.

With functionalization, functionalized nanoPS can be further conjugated with various desired molecules, which are of interest for a variety of applications, such as biomolecules, small molecules, therapeutic agents, micro- and nanoparticles, pharmaceutically active moieties, macromolecules, diagnostic labels, chelating agents, dispersants, charge modifying agents, viscosity modifying agents, surfactants, coagulation agents and flocculants, as well as various combinations of these chemical compounds.

Known methods for polysaccharide functionalization or derivatization can be used. For example, one approach is the introduction of carbonyl groups, by selective oxidation of glucose hydroxyl groups at positions of C-2, C-3, C-4 and/or C-6. There is a wide spectrum of oxidative agents which can be used such as periodate (e.g., potassium periodate), bromine, dimethyl sulfoxide/acetic anhydride (DMSO/Ac₂O) [e.g., U.S. Pat. No. 4,683,298], Dess-Martin periodinane, etc.

nanoPS functionalized with carbonyl groups are readily reactive with compounds bearing primary or secondary amine groups. This results in imine formation which can be further reduced to amine with a reductive agent e.g., sodium borohydrate. Thus, the reduction step provides an amino-product that is more stable than the imine intermediate, and also converts unreacted carbonyls in hydroxyl groups. Elimination of carbonyls significantly reduces the possibility of non-specific interactions of derivatized nanoparticles with non-targeted molecules, e.g. plasma proteins.

The reaction between carbonyl- and amino-compounds and the reduction step can be conducted simultaneously in one vessel (with a suitable reducing agent introduced to the same reaction mixture). This reaction is known as direct reductive amination. Here, any reducing agent, which selectively reduces imines in the presence of carbonyl groups, e.g., sodium cyanoborohydrate, can be used.

For the preparation of amino-functionalized nanoPS from carbonyl-functionalized nanoPS, any ammonium salt or primary or secondary amine-containing compound can be used, e.g., ammonium acetate, ammonium chloride, hydrazine, ethylenediamine, or hexanediamine. This reaction can be conducted in water or in an aqueous polar organic solvent e.g., ethyl alcohol, DMSO, or dimethylformamide.

Reductive amination of nanoPS can be also achieved by using the following two step process. The first step is allylation, i.e., converting hydroxyls into allyl-groups by reaction with allyl halogen in the presence of a reducing agent, e.g., sodium borohydrate. In the second step, the allyl-groups are reacted with a bifunctional aminothiol compound, e.g., aminoethanethiol [3,4].

Amino-functionalized nanoPS is an important product which are amendable to further modification. For example, amino groups are reactive to carbonyl compounds (aldehydes and ketones), carboxylic acids and their derivatives, (e.g., acyl chlorides, esters), succinimidyl esters, isothiocyanates, sulfonyl chlorides, etc.

In certain embodiments, nanoPS molecules are functionalized using the process of cyanylation. This process results in the formation of cyanate esters and imidocarbonates on polysaccharide hydroxyls. These groups react readily with primary amines under very mild conditions, forming covalent linkages. Cyanylation agents such as cyanogen bromide, and, preferably, 1-cyano-4-diethylamino-pyridinium (CDAP), can be used for functionalization of the nanoPS molecules [5].

Functionalized nanoPS can be directly attached to a chemical compound bearing a functional group that is capable of binding to carbonyl- or amino-groups. However, for some applications it may be important to attach chemical compounds via a spacer or linker including for example a polymer spacer or a linker. These can be homo- or hetero-bifunctional linkers bearing functional groups which include, but are not limited to, amino, carbonyl, sulfhydryl, succimidyl, maleimidyl, and isocyanate e.g., diaminohexane, ethylene glycobis(sulfosuccimidylsuccinate) (sulfo-EGS), disulfosuccimidyl tartarate (sulfo-DST), dithiobis(sulfosuccimidylpropionate) (DTSSP), aminoethanethiol, and the like.

### Chemical Compounds and Modifiers for NanoPS/Conjugation

In certain embodiments, chemical compounds which can be used to modify nanoPS include, but are not limited to: biomolecules, small molecules, therapeutic agents, micro- and nanoparticles, pharmaceutically active moieties, macromolecules, diagnostic labels, chelating agents, dispersants, charge modifying agents, viscosity modifying agents, surfactants, coagulation agents and flocculants, as well as various combinations of these chemical compounds.

In certain embodiments, biomolecules used as chemical compounds to modify nanoPS include, but are not limited to, enzymes, receptors, neurotransmitters, hormones, cytokines, cell response chemical compounds such as growth factors and chemotactic factors, antibodies, vaccines, haptens, toxins, interferons, ribozymes, anti-sense agents, and nucleic acids.

In certain embodiments, small molecule chemical compounds used to modify nanoPS result in functionalized nanoPS that is useful for pharmaceutical applications and include, but are not limited to, vitamins, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics and imaging agents.

In certain embodiments, small molecule modifiers of nanoPS can be those which can be useful as catalysts and include, but are not limited to, metal-organic complexes.

In certain embodiments, pharmaceutically useful moieties used as modifiers for nanoPS include, but are not limited to, hydrophobicity modifiers, pharmacokinetic modifiers, biologically active modifiers and detectable modifiers.

In certain embodiments, nanoPS can be modified with chemical compounds which have light absorbing, light emitting, fluorescent, luminescent, Raman scattering, fluorescence resonant energy transfer, and electroluminescence properties.

In certain embodiments, diagnostic labels of nanoPS include, but are not limited to, diagnostic radiopharmaceutical or radioactive isotopes for gamma scintigraphy and positron emission tomography (PET), contrast agents for Magnetic Resonance Imaging (MRI) (e.g. paramagnetic atoms and superparamagnetic nanocrystals), contrast agents for computed tomography, contrast agents for imaging with X-rays, contrast agents for ultrasound diagnostic methods, agents for neutron activation, and other moieties which can reflect, scatter or affect X-rays, ultrasounds, radiowaves and microwaves, fluorophores in various optical procedures, etc. Diagnostic radiopharmaceuticals include gamma-emitting radionuclides, e.g., indium-111, technetium-99m and iodine-131, etc. Contrast agents for MRI (Magnetic Resonance Imaging) include magnetic compounds, e.g. paramagnetic ions, iron, manganese, gadolinium, lanthanides, organic paramagnetic moieties and superparamagnetic, ferromagnetic and antiferromagnetic compounds, e.g., iron oxide colloids, ferrite colloids, etc. Contrast agents for computed tomography and other X-ray based imaging methods include compounds absorbing X-rays, e.g., iodine, barium, etc. Contrast agents for ultrasound based methods include compounds which can absorb, reflect and scatter ultrasound waves, e.g., emulsions, crystals, gas bubbles, etc. Other examples include substances useful for neutron activation, such as boron and gadolinium. Further, labels can be employed which can reflect, refract, scatter, or otherwise affect X-rays, ultrasound, radiowaves, microwaves and other rays useful in diagnostic procedures. In certain embodiments a modifier comprises a paramagnetic ion or group.

In certain embodiments, two or more different chemical compounds are used to produce multifunctional derivatives. For example, the first chemical compound is selected from a list of potential specific binding biomolecules, such as antibody and aptamers, and then the second chemical compound is selected from a list of potential diagnostic labels.

In certain embodiments, nanoPS molecules can be used as templates for the preparation of inorganic nanomaterials using methods that are generally known in the art (see, for example, Mirkin and Niemeyer, as cited above). This can include functionalization of nanoPS with charged functional groups, followed by mineralization which may include incubation of functionalized nanoPS in solutions of various cations, e.g. metals, semiconductors. Mineralized nanoPS can be then purified and used in various applications, which include but are not limited to medical diagnostics, sensors, optics, electronics, etc.

The present description is further expanded with reference to the following non-limiting working examples.

### Example 1

### Fermentation of G. sulfurreducens PCA

G. sulfurreducens PCA (ATCC 51573) was grown under strict anaerobic conditions at 30° C. for 48 h in modified NBAF prepared according to [7]. The medium contained 15 mM of sodium acetate as electron-donor and 40 mM of sodium fumarate as an electron acceptor. Fermentation was carried out in 15 L vessels, each containing 10 L of the medium. The fermentation process in each vessel was started with one liter of a 24 hour old seed culture. Bacterial cells were harvested by centrifugation at 8,000×g for 15 min and stored at −20° C. The yield was 0.22-0.25 g of cell dry wt per liter of the growth medium.

### Example 2

### Isolation of Polysaccharide Nanoparticles from the Biomass of G. Sulfurreducens PCA by Using Method #1

Method #1 comprises the following steps:
a) mixing bacterial biomass with a suitable amount of water to produce a suspension with a final biomass concentration of 10-80 g of dry wt./L, preferably 40 g/L;
b) adding 90% (w/v) aqueous phenol to the suspension of bacterial cells to produce a final phenol concentration of 30-50%, preferably 45%. This is followed by vigorous stirring and heating of the suspension to a temperature of 50-68° C., preferably for 10-15 min;
c) cooling the mixture to about 0-5° C.;
d) centrifuging the suspension (4000-6000×g for 10-20 min at 4° C.), collecting the water fraction, and discarding the insoluble pellet;
e) diluting the phenol fraction from step d with pure water by 25-40% (v/v), and repeating steps b, c, and d, pooling the collected water fractions;
f) removing the phenol from the collected water fractions by dialyzing against pure water using a membrane with a 12-14 kilodalton molecular weight cut-off for 48-72 h at room temperature;
g) adjusting the pH to 8.0 with 0.5M Tris.HCl buffer (pH 8.0), adding magnesium chloride up to 2 mM, adding DNAse and RNAse, at final enzyme concentrations of 200 μg/ml and 50 μg/ml respectively, stirring at 37° C. for 1-3 h, centrifuging (50,000×g for 45 min at 4° C.), and collecting the supernatant;
h) centrifuging again (200,000×g for 3 h at 4×C), collecting the pellet;
i) adding SDS and Na-EDTA to have final concentrations of 2% and 0.1 M, respectively, adjusting the pH of the mixture to 8.5-9.5 with 0.5M NaOH followed by adding proteinase K (50 μg/ml), stirring at 60° C. for 2 h;
j) dialyzing against pure water for 24-72 h, and freeze drying;
k) dissolving the dried material from the previous step in a 0.5 M solution of magnesium chloride to the final solid/liquid ratio of 1/5-1/6 (wt/vol), centrifuging at 16,000×g for 20 min at 4° C.;
l) dialyzing the supernatant for 72 h against water and freeze-drying 400 g (wet wt.) of biomass was produced using the procedure in Example 1 and it was placed in a 5 L round bottom glass vessel and suspended in 1.5 L of nanopure water. Then 1.5 L of 90% (w/v) aqueous phenol was added to the suspension. This was followed by vigorous stirring and heating of the suspension to a temperature of 68° C. After 15 min of stirring, the mixture was cooled to about 0° C. using an ice bath and was centrifuged at 6000×g at 4° C.

The pellet, containing insoluble cell debris was discarded. The supernatant contained two layers: a water fraction and a phenol fraction. The water fraction was collected and kept at 4° C. for further use, while the phenol fraction was re-extracted with ⅓rd volume of pure water under the conditions described above. This operation was repeated 3 times before the phenol fraction was discarded. All collected water fractions were pooled and dialyzed against nanopure water using a membrane with a 12-14 kilodalton molecular weight cut-off for 48-72 h at room temperature.

Dialysate was supplemented with magnesium chloride (MgCl₂) to make a final concentration of 2 mM, and the pH was adjusted to 8.0 with a 0.5M Tris.HCl buffer. Then the mixture was treated with DNAse and RNAse at final enzyme concentrations of 200 μg/ml and 50 μg/ml respectively. The mixture was stirred at 37° C. for 3 h and then centrifuged at 50,000×g for 45 min at 4° C., collecting the supernatant. The supernatant was centrifuged again at 200,000×g for 3 h at 4° C., collecting the pellet. The pellet was then resuspended in 2% (w/v) SDS in 0.1M Na₂-EDTA, and the pH of the mixture was adjusted to 8.5-9.5 using 0.5M NaOH. Proteinase K (25 μg/ml, final concentration) was added to the mixture and it was stirred at 60° C. for 2 h. Then the mixture was dialyzed against nanopure water for 24-72 h at room temperature, changing the water every 12 h. The dialysate was freeze-dried.

The lyophilized material was dissolved in a 0.5 M solution of magnesium chloride at a final solid/liquid ratio of 1/6 (wt/vol). The mixture was cooled in the fridge at 4° C. for 24 h and then it was centrifuged at 16,000×g for 20 min. The supernatant was dialyzed for 72 h as described above and freeze-dried. This method yielded 15 g (dry wt) of nanoPS.

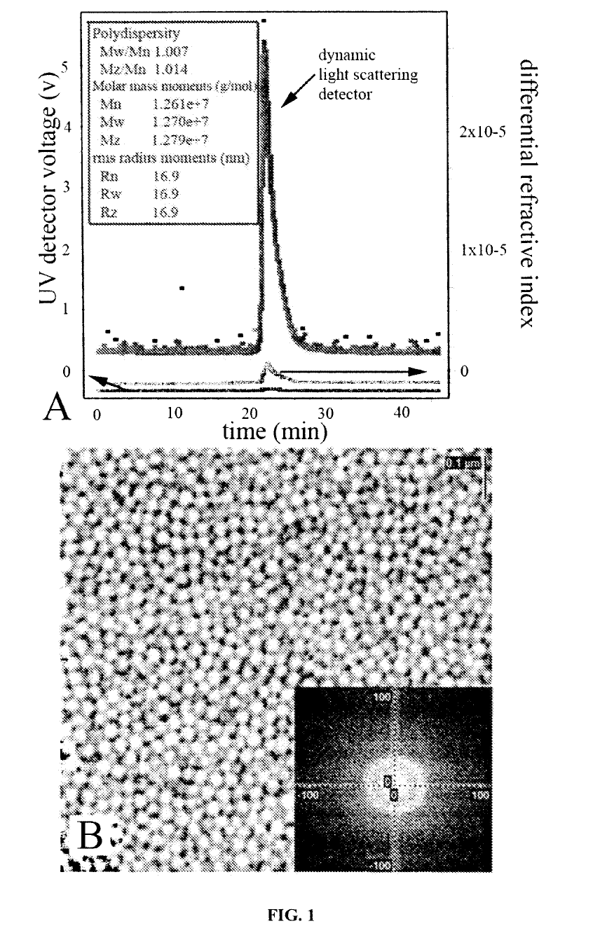

The weight average molar mass moment Mw and polydispersity index (Mw/Mn) of the resultant polysaccharide nanoparticles were 1.270×10̂7 and 1.007 as measured using Size Exclusion Chromatography (See FIG. 1A). A Waters chromatography system equipped with a Phenomenex BioSep S4000 column and three detectors (UV absorption, differential refractive index and multiple angle laser light scattering (MALLS)) was used.

The diameter and size polydispersity of the resultant polysaccharide nanoparticles were 33.3 nm and 18.2% respectively, as measured using Atomic Force Microscopy (AFM, see FIG. 1B). To perform AFM measurements the nanoparticles were dissolved in ultrapure water (1.0 mg/ml), then aliquots were dried onto a freshly cleaved mica substrate (approximately 1×1 cm). The AFM images were collected using tapping mode.

The mean diameter and size polydispersity of the resultant polysaccharide nanoparticles were 40.2 nm and 3.5% respectively, as measured using a Brookhaven BI-200SM Dynamic Light Scattering system equipped with a TurboCorr correlator (see FIG. 1C).

### Example 3

### Isolation of Polysaccharide Nanoparticles from the Biomass of G. Sulfurreducens PCA Using Method #2

Method #2 for the isolation of polysaccharide nanoparticles from the microbial biomass comprises the following steps:

a) resuspending the biomass in a solution of 50 mM Tris.HCl (pH 8.0), adding magnesium chloride up to 2 mM, adding DNAse and RNAse (100 μg/ml and 25 μg/ml respectively), stirring for 15-30 min at 37° C. to reduce the viscosity;

b) disrupting the microbial cells using a French press (at 15,000 lb/in2);

c) adding DNAse and RNAse (to achieve final enzyme concentrations of 200 μg/ml and 50 μg/ml respectively), stirring for 15-30 min at 37° C.;

d) centrifuging (21,000×g for 2 h at 4° C.), collecting the supernatant, discarding the pellet containing cell walls, insoluble proteins etc.;

e) adding SDS and Na-EDTA to a final concentration of 2% (w/v) and 0.1 M respectively, bringing the pH to 9.0 with 0.1 M NaOH, and treating with proteinase K (up to 200 μg/ml), for 2 h at 60° C.); and dialyzing using a membrane with a 12-14 kilodalton molecular weight cut-off against water to remove proteins and lipids;

f) adding 3 volumes of a solution of magnesium chloride in 95% (v/v) ethanol (0.375 M), stirring and cooling to 0-4° C.; centrifuging (16,000×g for 30 min at 4° C.); keeping the pellet containing nanoPS and LPS;

g) dissolving the pellet in 2% SDS in 0.1 M Na-EDTA (pH 7.5), dialyzing using a membrane with a 12-14 kilodalton molecular weight cut-off against pure water;

h) adding calcium chloride to the solution to a final concentration of 0.1-0.2M, and adding ethanol up to 10% (v/v), and centrifuging (16,000×g for 30 min at 4° C.); keeping the supernanant and discarding the pellet containing LPS;

j) adding ethanol or another appropriate solvent to the supernatant to achieve a final solvent concentration in the range of 50-80%; cooling the mixture to 0-4° C., centrifuging (16,000×g for 20 min at 4° C.);

k) resuspending the pellet containing polysaccharide nanoparticles in water and dialyzing using a membrane with a 12-14 kilodalton molecular weight cut-off against pure water;

l) freeze drying of the solution containing polysaccharide nanoparticles to produce a powder.

400 g (wet wt.) of biomass from Example 1 was resuspended in 50 mM TRIS.HCl solution, pH 8.0, supplemented with magnesium chloride to a final concentration of 2 mM, DNAse and RNAse (100 μg/ml and 25 μg/ml respectively), and stirred for 15-30 min at room temperature. Then bacterial cells were disrupted using a French press (at 15,000 lb/in2).

DNAse and RNAse were added to a cell homogenate to achieve final enzyme concentrations of 200 μg/ml and 50 respectively, followed by stirring for 2 h at 37 oC. The mixture was centrifuged (16,000×g for 20 min at 4° C.) and the pellet was discarded.

SDS and Na₄-EDTA were added to the supernatant to produce final concentrations of 2% (w/v) and 0.1 M respectively, then proteinase K (50 μg/ml was added and the solution was stirred for 2 h at 60° C. The mixture was centrifuged (50,000×g for 2 hours at 20° C.) and the pellet was discarded.

The solution was then dialyzed using a membrane with a 12-14 kilodalton molecular weight cut-off against nanopure water for 48 h. The dialyzed solution was mixed with 3 volumes of pre-cooled 0.375 M solution of magnesium chloride in 95% (w/v) ethanol, stirred and cooled to 4° C. using an ice bath. The resulting solution was then centrifuged (16,000×g for 20 min at 4° C.), the pellet was dissolved in 2% SDS in 0.1 M Na₄-EDTA and dialyzed using a membrane with a 12-14 kilodalton molecular weight cut-off against pure water. Calcium chloride was added to the dialysate to achieve a final concentration of 0.2M CaCl₂ and ethanol up to 10% (v/v), the mixture was left in the fridge for 24 h and then it was centrifuged (at 16,000×g for 20 min at 4° C.); the supernanant was retained and the pellet containing LPS was discarded. The supernatant was mixed with 3 volumes of 95% (w/v) ethanol, cooled to 0° C., and centrifuged (16,000×g for 20 min at 4° C.). The pellet was resuspended in water and dialyzed using a membrane with a 12-14 kilodalton molecular weight cut-off against pure water. The dialysate was freeze-dried to produce a powder of the polysaccharide nanoparticles. The yield was 12.2 g of (dry wt.) of polysaccharide nanoparticles.

The weight average molar mass moment M_{w} and polydispersity index (M_{w}/Mₙ) of the resultant nanoparticles were 5.362×10̂6 and 1.031 as measured using Size Exclusion Chromatography. A Waters chromatography system equipped with a Phenomenex BioSep S4000 column and three detectors (UV absorption, differential refractive index and multiple angle laser light scattering (MALLS)) was used.

The diameter and size polydispersity of the resultant polysaccharide nanoparticles were 35.3 and 22.7%. respectively, as measured using Atomic Force Microscopy (AFM, see FIG. 1B). To perform AFM measurements the nanoparticles were dissolved in ultrapure water (1.0 mg/ml), then aliquots were dried onto a freshly cleaved mica substrate (approximately 1×1 cm). The AFM images were collected using tapping mode.

The mean diameter and size polydispersity of the resultant nanoparticles were 60.2 nm and 43.7% respectively as measured using a Wyatt DynaPro Titan Dynamic Light Scattering system.

### Example 4(a)

### Fermentation of Escherichia coli K12

E. coli K12 was grown under aerobic conditions at 32° C. for 16 h in a synthetic medium containing 10 g/L of dextrose and 1 g/L of ammonium sulfate as the sole nitrogen source [6]. Fermentation was carried out in a 15 L fermentor vessel, containing 10 L of the medium with agitation at 200 rpm. The fermentation process was started with 100 ml of a 12 hour old seed culture. Bacterial cells were harvested by centrifugation at 6,000×g for 15 min and transferred into in a 15 L fermentor vessel, containing 10 L of fresh synthetic medium of the same composition as previously described except that the nitrogen source (ammonium sulfate) was excluded. The fermentation continued under the same conditions for 6 h and then bacterial cells were harvested by centrifugation at 8,000×g for 15 min and stored at −20° C.

### Example 4(b)

### Isolation of Polysaccharide Nanoparticles from the Biomass of Escherichia coli K12

E. coli K12 was grown in a synthetic medium containing 20 g/L of dextrose, 2.5 g/L of ammonium sulfate as the sole nitrogen source, 1.5 g of K₂HPO₄, 0.6 g of KH₂PO₄, 0.2 g magnesium sulfate and 10 mg of thiamine per liter. One liter of medium was supplemented with 5 mL of a trace element solution containing 1 mol of HCl, 1.5 g of MnCl₂ 4H₂O, 1.0 g of ZnSO₄, 0.3 g of H3BO₃, 0.25 g of Na₂MoO₄ 2H₂O, 0.15 g of CuCl₂ 2H₂O, 0.85 g of Na₂EDTA 2H₂O, 4.0 g of CaCl₂ 2H₂O and 4.5 g of FeSO₄ 7H₂O per liter. Cultivation was carried out in a 1.5 L fermentation vessel, containing 1.0 L of the medium at 32° C. and constant aeration. The dissolved oxygen concentration was maintained at a minimum of 20% by controlling agitation and air flow rate. A sodium hydroxide solution was used to maintain the pH at 7.2 The fermentation process was started with 50 ml of a 12 hour old seed culture. Bacterial cells were harvested at the early stationary growth phase by centrifugation at 6,000×g for 15 min and transferred into a 15 L fermentor vessel, containing 10 L of fresh synthetic medium of the same composition as previously described except that the nitrogen source (ammonium sulfate) was excluded. The fermentation continued under the same conditions for 6 h and then bacterial cells were harvested by centrifugation at 8,000×g for 15 min and freeze dried. The biomass yield was 2.75 g of dry wt. The biomass was ground using a mortar and pestle, resuspended in 100 ml of water and then processed under the conditions described in Example 2. The yield of polysaccharide nanoparticles was 0.25 g (dry wt).

The mean diameter and size polydispersity of the resultant polysaccharide nanoparticles were 40.8 nm and 14.3% respectively, as measured using a Wyatt DynaPro Titan Dynamic Light Scattering system.

### Example 5

### Isolation of nanoPS from Oysters

The oysters were obtained from local grocery store. 100 g of oyster tissue (wet wt) was homogenized in a blender and processed as described in Example 2. The yield of polysaccharide nanoparticles was 1.25 g (dry wt).

The weight average molar mass moment M_{w} and polydispersity index (M_{w}/Mₙ) of the resultant polysaccharide nanoparticles extracted from oysters were 2.267×10̂7 and 1.099 as measured using Size Exclusion Chromatography. A Waters chromatography system equipped with a Phenomenex BioSep 54000 column and three detectors (UV absorption, differential refractive index and multiple angle laser light scattering (MALLS)) was used.

The mean diameter and size polydispersity of the resultant polysaccharide nanoparticles extracted from oysters were 60.4 nm and 30.9% respectively, as measured using a Wyatt DynaPro Titan Dynamic Light Scattering system.

### Example 6

### Characterization of nanoPS from Examples 2, 3, and 5

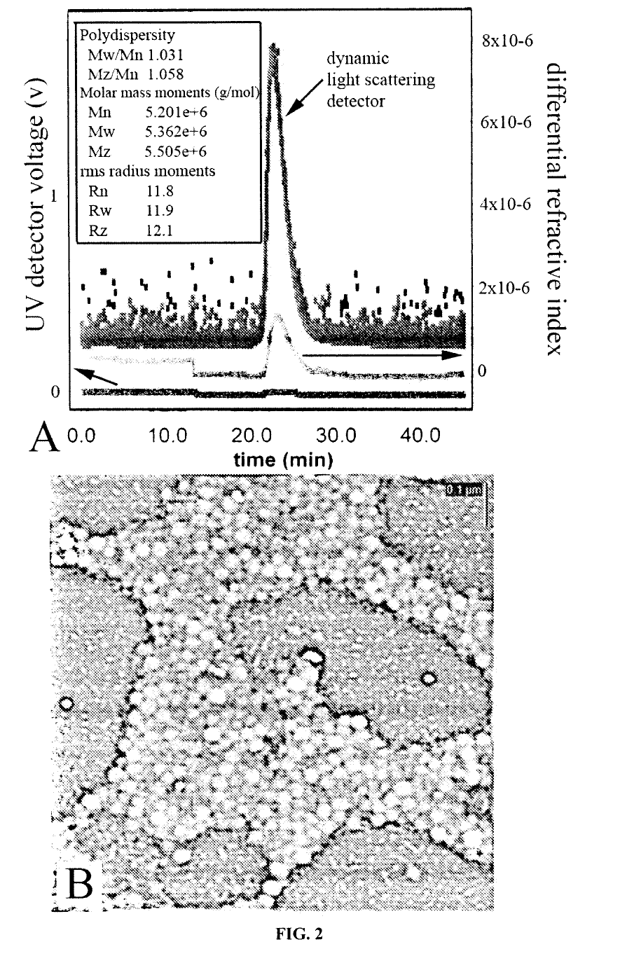

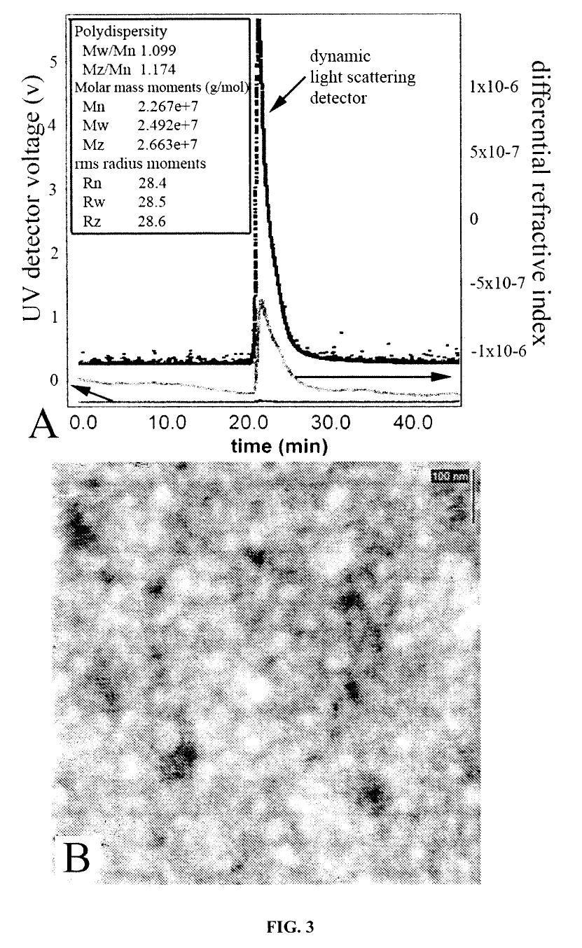

The nanoPS molecules were dissolved in 0.01M KNO₃ and analyzed using a size exclusion chromatography unit equipped with a Phenomenex BioSep S4000 column and three detectors (UV absorption, differential refractive index and multi-angle dynamic laser light scattering (MALLS)). The results are shown in FIGS. 1, 2, and 3.

The material was also analyzed using atomic force microscopy (AFM, Dimension 3100 AFM, Veeco Instruments Corp., Santa-Barbara, Calif.) operating in tapping mode using standard silicon cantilevers (AC160TS, force constant 42 N/m, resonance frequency 300 kHz, Al back coating, Olympus, Tokyo, Japan). The nanoPS preparations were dissolved in nanopure water (1 mg/ml). Then aliquots were dried onto a freshly cleaved mica substrate (approximately 1×1 cm). Representative AFM images are shown on FIGS. 1B, 2B, and 3B. The size of the nanoPS molecules prepared in examples 2 and 3 was determined to be 33.3 with size polydispersity 18.2%, and 35.3 with size polydispersity 22.7%.

Chemical characterization of the structure of the nanoPS molecules was performed using gas chromatography mass spectrometry (GC-MS, PolarisQ GC-MS FID, Thermo Finnigan, Austin, Tex.) and nuclear magnetic resonance spectroscopy (NMR, Bruker 400 MHz spectrometer). All analysis was perfomed using D₂O as a solvent.

The sugar composition was analyzed using the alditol-acetate method (GC-MS), and this revealed that nanoPS is a glucose homopolymer.

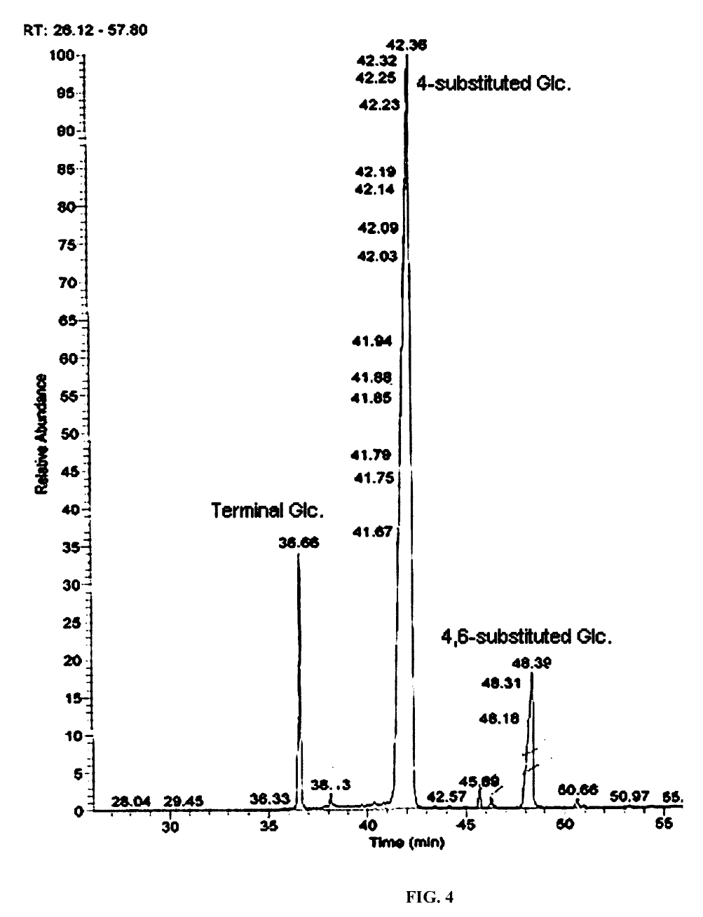

Permethylated alditol acetate derivatives were used for linkage analysis (GC-MS, electron impact mode). The glucose residues are mainly linked through a 1→4 type linkage and branching occurs predominantly at position 6. The approximate ratios for the terminal, 1→4 and 1→4,6 linked glucose residues are:
nanoPS isolated in Example 2:1:12.7:1.3 respectively.
nanoPS isolated in Example 3:1:11.5:0.8 respectively (See FIG. 4)

Proton NMR revealed one major anomeric peak at 5.41 ppm (a 1→4) and a minor one at 5.02 ppm (α1→4,6). The pattern of the ring region is indicative of a large structure.

NOESY NMR experiments suggested an extremely densely packed molecular structure.

### Example 7

### Isolation of Polysaccharide Nanoparticles from the Biomass of Greenshell Mussels (Perna canaliculus) (New Zealand)

1302 g wet wt. (equal to 257.8 g dry wt.) of Greenshell mussel meat from a local grocery store was mixed with 2.5 L of pure water and homogenized in a blender at 4° C. for 5 min to an average particle size less than 1 mm.

The homogenate was centrifuged at 8000×g at 4° C. and the supernatant (2.5 L) was transferred to a 5 L round bottom glass vessel. Then 0.8 L of 90% (w/v) aqueous phenol was added to the supernatant. This was followed by vigorous stirring and raising the temperature of the suspension to 68° C. After stirring at this temperature for 15 min. the mixture was cooled to about 4° C. in a refrigerator overnight. Then the water fraction was collected, while the phenol fraction was discarded.

The water fraction was centrifuged at 8000×g, at 4° C. and the pellet was discarded. Then ethanol was added to the supernatant to a final concentration of 60%, and the mixture was cooled to 4° C. The precipitate was isolated by centrifugation (at 6000×g, at 4° C.), resuspended in 0.4 L of water and dialyzed against pure water using a 12-14 kDa molecular weight cut-off membrane for 48-72 hrs at room temperature, changing the water every 12 hours.

The dialysate was supplemented with magnesium chloride to make a final 2 mM MgCl₂ concentration, treated with DNAse and RNAse, at final enzyme concentrations of 25 μg/ml and 15 μg/ml respectively, at pH 8.0, adjusted with 0.5M Tris*HCl buffer. The mixture was stirred at 37° C. for 3 hrs, then SDS and Na-EDTA were added to have final concentrations of 2% (w/v) and 0.1 M respectively. The mixture was treated with proteinase K (12 μg/ml) at pH 8.5-9.5, adjusted with 0.5M NaOH, under stirring at 60° C. for 2 hours. Then the mixture was dialyzed against pure water for 24-72 hrs at room temperature, changing the water every 12 hours. The dialysate was freeze-dried.

The yield of polysaccharide nanoparticles was 29.7 g (dry wt) which corresponds to 11.2% of the mussel meat dry weight.

The weight average molar mass moment M_{w} and polydispersity index (M_{w}/Mₙ) of the resultant polysaccharide nanoparticles extracted from Greenshell mussels were 1.444×10̂7 and 1.086 as measured using Size Exclusion Chromatography. A Waters chromatography system equipped with a Phenomenex BioSep S4000 column and three detectors (UV absorption, differential refractive index and multiple angle laser light scattering (MALLS)) was used.

The mean diameter and size polydispersity of the resultant polysaccharide nanoparticles extracted from Greenshell mussels were 29.7 nm and 3.8% respectively, as measured using a Brookhaven BI-200SM Dynamic Light Scattering system equipped with a TurboCorr correlator.

### Example 8

### Conjugation of Polysaccharide Nanoparticles with 5-Aminofluorescein Using Cyanylation Chemistry

300 mg of polysaccharide nanoparticles produced according to Example 5 was dissolved in 15 ml of pure water and cooled to 4° C. Using 10% sodium carbonate (Na₂CO₃), the pH of the solution was adjusted to 10.8. Then 80 mg of cyanogen bromide (CNBr) in 1 ml of dimethylformamide was added, the mixture was stirred and the pH was maintained at 10.8. After 4 minutes of stirring, the pH was adjusted to 8.5 with 20% acetic acid and 10 mg of 5-aminofluorescein in 1 ml of dimethylformamide was added. The mixture was stirred at RT for 4 hours in the dark. The nanoPS-aminofluorescein conjugate was precipitated from the reaction mixture with 3 volumes of cold (0° C.) ethanol. The precipitate was removed from the solution by centrifugation at 12000×g at 4° C. for 15 min. The pellet was resuspended in 5 ml of water and ethanol precipitation was repeated another 5 times. Then the product was lyophilized.

Analysis of the conjugate showed the following: from an assay, we measured 14 mg of aminofluorescein per 1 g of polysaccharide nanoparticles; the absorbance maximum occurred at 490.5 nm and the fluorescence emission maximum occurred at 517 nm (in a 0.05M potassium phosphate buffer, pH 7.0).

The mean diameter and size polydispersity of the resultant modified polysaccharide nanoparticles were 48.6 nm and 5.4% respectively, as measured using a Brookhaven BI-200SM Dynamic Light Scattering system equipped with a TurboCorr correlator.

### Example 9

### Conjugation of Polysaccharide Nanoparticles with Doxorubicin Using Cyanylation Chemistry 250 mg of polysaccharide nanoparticles produced according to Example 5 was dissolved in 10 ml of pure water and cooled to 4° C. Using 10% sodium carbonate (Na₂CO₃), the pH of the solution was adjusted to 10.8. Then 65 mg of cyanogen bromide (CNBr) in 1 ml of DMSO was added, the mixture was stirred and the pH was maintained at 10.8. After 4 minutes of stirring, the pH was adjusted to 8.5 with 20% acetic acid and 10 mg of doxorubicin hydrochloride in 1 ml of DMSO was added. The mixture was stirred at RT for 4 hours in the dark. The nanoPS-doxorubicin conjugate was precipitated from the reaction mixture with 3 volumes of cold (0° C.) ethanol. The precipitate was removed from the solution by centrifugation at 12000×g at 4° C. for 15 min. The pellet was resuspended in 5 ml of water and ethanol precipitation was repeated another 5 times. Then the product was lyophilized.

Analysis of the conjugate showed the following: from an assay, we measured 15 mg of doxorubicin per 1 g of polysaccharide nanoparticles; the absorbance maximum occurred at 480 nm (in a 0.05M potassium phosphate buffer, pH 7.0).

The mean diameter and size polydispersity of the resultant modified polysaccharide nanoparticles were 53.3 nm and 55.2% respectively, as measured using a Brookhaven BI-200SM Dynamic Light Scattering system equipped with a TurboCorr correlator.

### Example 10

### Periodate Oxidation of Polysaccharide Nanoparticles

Polysaccharide nanoparticles (1.0 g), produced according to Example 5, was dissolved in 100 ml of a 0.2M potassium phosphate buffer, pH 7.0, and 0.3 g of sodium periodate in 50 milliliters of water was added to the solution. The resulting mixture was stirred at room temperature for 2 h. Next 5 ml of ethylene glycol was added to quench the reaction. Then the solution was dialyzed against nanopure water, using a membrane with a 12-14 kilodalton molecular weight cut-off, for 24 h at room temperature. The resulting solution was lyophilized. The yield was of 0.81 g, and with the above conditions approximately 5% of the glucose residues were oxidized.

The mean diameter and size polydispersity of the resultant modified polysaccharide nanoparticles were 33.8 nm and 30.7% respectively, as measured using a Wyatt DynaPro Titan Dynamic Light Scattering system.

However, water solutions of oxidized polysaccharide nanoparticles were not stable. After two weeks of storage in water at 4° C., more than 85% of oxidized polysaccharide nanoparticles were hydrolyzed as measured using Dynamic Light Scattering.

### Example 11

### Conjugation of Oxidized Polysaccharide Nanoparticles with 5-Aminofluorescein

50 mg of oxidized polysaccharide nanoparticles from Example 10 was dissolved in 4 ml of 0.2 potassium phosphate buffer, pH 7.4. Then 1 ml of 0.5% (w/v) solution of 5-aminofluorescein in 50% (v/v) aqueous ethanol was added. The mixture was stirred at room temperature for 48 h in the dark. The polysaccharide nanoparticle—aminofluorescein conjugate was precipitated from the reaction mixture with 3 volumes of cold (0° C.) ethanol. The precipitate was removed from solution by centrifugation at 12000×g for 15 min at 4° C. The pellet was resuspended in 5 ml of water and the ethanol precipitation procedure was repeated 4 times, until all of the unreacted aminofluorescein was washed away, as monitored by the supernatant absorbance at 487 nm. The washed pellet was resuspended in 5 ml of 0.2M potassium phosphate buffer, pH 7.4, and sodium borohydride was added to the solution to reach a final concentration of 1 mg/ml. The solution was stirred for 15 min and the nanoPS-aminofluorescein conjugate was precipitated as described above. The final product was lyophilized.

Analysis of the conjugate showed the following: Aminofluorescein:glucose ratio of 1:233; absorbance maximum at 490.5 nm; and fluorescence emission maximum at 517 nm (in a 0.05M potassium phosphate buffer, pH 7.0).

The mean diameter and size polydispersity of the resultant modified polysaccharide nanoparticles were 72.8 nm and 31.7% respectively, as measured using a Wyatt DynaPro Titan Dynamic Light Scattering system.

However, similarly to oxidized polysaccharide nanoparticles from Example 10, water solutions of the 5-aminofluorescein modified polysaccharide nanoparticles were not stable, and after several weeks of storage in water at 4° C. most of the polysaccharide nanoparticles were hydrolyzed as measured using Dynamic Light Scattering.

### Example 12

### Conjugation of Oxidized nanoPS with 8-Aminonaphthalene-1,3,6-trisulfonic Acid (ANTS)

50 mg of the oxidized nanoPS of Example 10 was dissolved in 4 ml of a 0.1 M of sodium borate, pH 8.5 and 1 ml of 1.0% (w/v) ANTS in water and then 1 ml of 2% (w/v) of sodium cyanoborohyride (NaCNBH₃) in the same buffer were added to the solution.

The mixture was stirred at 45° C. for 12 h in the dark. The nanoPS-ANTS conjugate was separated from the reaction mixture and washed as was described in Example 8, with the exception that the ANTS concentration in the supernatant was monitored by absorbance at 351 nm. The final product was lyophilized.

Analysis of the conjugate showed the following: ANTS:glucose ratio of 1:140; absorbance maximum at 354 nm (UV mini 1240 UV-VIS spectrophotometer, Shimadzu, Kyoto, Japan); and fluorescence emission maximum at 520 nm in a 0.05M potassium phosphate buffer, pH 7.0 (PTI QuantaMaster UV VIS spectrofluorometer, Photon Technology International Inc., London, Canada)

### Example 13

### Conjugation of Oxidized nanoPS with Congo Red

50 mg of oxidized nanoPS of Example 10 was dissolved in 4 ml of a 0.2 potassium phosphate buffer, pH 7.4 and 1 ml of 1.0% (w/v) aqueous solution of Congo Red was added to it. The mixture was stirred at room temperature for 48 h in the dark. The nanoPS-Congo Red conjugate was precipitated from the reaction mixture with 3 volumes of cold (0° C.) ethanol. The precipitate was removed from solution by centrifugation at 12,000×g for 15 min at 4° C. The pellet was resuspended in 5 ml of water and the ethanol precipitation procedure was repeated 4 times, until all of the unreacted Congo Red was washed away, as monitored by the supernatant absorbance at 487 nm. The washed pellet was resuspended in 5 ml of 0.2M potassium phosphate buffer, pH 7.4, and sodium borohydride was added to achieve a final concentration of 1 mg/ml. After 15 min of stirring, the nanoPS-Congo Red conjugate was precipitated as described above. The final product was lyophilized. The conjugate yield was 49 mg.

Analysis of the conjugate showed the following: Congo Red:glucose ratio of 1:1300; absorbance maximum at 486.5 nm; and fluorescence emission maximum at 580 nm (in a 0.05M potassium phosphate buffer, pH 7.0).

### Example 14

### Amination of Polysaccharide Nanoparticles

200 mg of polysaccharide nanoparticles, produced according to Example 5, was dissolved in 2 ml of DMSO and 250 mg dry, powdered NaOH was added to the solution. After 15 min. of stirring, 1.5 ml of 2-bromoethylamine hydro-chloride was added to the reaction mixture (216.66 mg/ml in DMSO). The reaction was allowed to proceed for 4 hrs with constant stirring. After 4 h, 10 ml water was added to the mixture and the aminated polysaccharide nanoparticles were precipitated from the solution with ethanol (2 volumes of ethanol were added, cooled to 0° C. and centrifuged at 12000×g for 15 min at 4° C.). The precipitate was placed in water (10 ml) and the ethanol precipitation step was repeated 3 more times. The sample was dried and the degree of substitution was estimated using proton NMR spectroscopy. According to the NMR data, 5.0 mol % of the glucose units were aminated (1 in every 20 sugars).

The mean diameter and size polydispersity of the resultant modified polysaccharide nanoparticles were 25.6 nm and 47.0% respectively, as measured using a Wyatt DynaPro Titan Dynamic Light Scattering system.

### Example 15

### Conjugation of Aminated Polysaccharide Nanoparticles with Fluorescamine

17 mg of aminated polysaccharide nanoparticles of Example 14, dissolved in 8 ml of DMSO was allowed to react with 200 μl of fluorescamine solution (50 mg/ml in acetone) for 30 min at RT. After 30 min, 16 ml of water and 300 μl of 1M CaCl₂ were added to the reaction and the polysaccharide nanoparticle conjugate was precipitated with 2 volumes of ethanol as described in Example 14. The ethanol precipitation step was repeated 3 more times (until the unreacted fluorescamine was washed away). All of the above steps were performed in the dark. The emission spectra for the polysaccharide nanoparticle-NH-fluorescamine conjugate was recorded using a PTI QuantaMaster UV-vis spectrofluorometer (Photon Technology International Inc., London, Canada) at an excitation wavelength of 386 nm (100 mM borate buffer, pH 8.5). The degree of conjugation was calculated as 0.9 mol % (1 in every 111 glucose units was conjugated), based on the 380 nm absorbance value (UV mini 1240 UV-vis spectrophotometer, Simadzu, Kyoto, Japan).

The mean diameter and size polydispersity of the resultant modified polysaccharide nanoparticles were 34.8 nm and 49.2% respectively, as measured using a Wyatt DynaPro Titan Dynamic Light Scattering system.

### Example 16

### Conjugation of Aminated Polysaccharide Nanoparticles with Rhodamine B

25 mg of aminated polysaccharide nanoparticles of Example 14 was dissolved in 5 ml of a 100 mM carbonate buffer pH 9.6 and 150 μl Rhodamine B isothiocyanate solution (100 mg/ml in DMSO) was added. After 120 min of stirring at RT, the solution was neutralized with HCl, and then it was diluted with an additional 5 ml of water and precipitated with ethanol as described in Example 14. The ethanol precipitation step was repeated 3 more times (until the free dye was washed away). The procedure was carried out in the dark. The degree of conjugation is 0.3 mol % (calculated from the absorbance value at 540 nm; UV mini 1240 UV-vis spectrophotometer, Simadzu, Kyoto, Japan). The rhodamine B conjugated polysaccharide nanoparticles were used to demonstrate polysaccharide nanoparticle uptake by normal murine endothelial cells (see Example 21).

The size distribution of the resultant modified polysaccharide nanoparticles was bi-modal, with one peak having a mean diameter and size polydispersity of 30.6 nm and 17.5% respectively, and the other peak having a mean diameter and size polydispersity of 124.4 nm and 20.0% respectively, as measured using a Wyatt DynaPro Titan Dynamic Light Scattering system.

### Example 17

### Conjugation of Polysaccharide Nanoparticles_with Nonenyl-Succinic Anhydride in Pyridine

220 mg of polysaccharide nanoparticles, produced according to Example 5, was made into a suspension in pyridine (three hours mixing at 50° C.) and 1.5 ml n-SA was added to it in 4 portions (during a one hour period). The reaction mixture was kept at 50° C., O/N (16 hrs) with stirring. The system was cooled to RT and 4 ml hexane was used to precipitate the product. The pellet was collected by centrifugation 5000×g for 15 min and re-suspended in hexane, then pelleted again using the same procedure and this step was repeated two more times. Finally the pellet was dried and then was placed in 15 ml of water and the pH was adjusted to 7.0 and lyophilized. The degree of substitution was calculated from proton NMR spectroscopy data as 3.5 mol % n-SA: nonenyl-succinic anhydride.

The mean diameter and size polydispersity of the resultant modified polysaccharide nanoparticles were 67.4 nm and 28.2% respectively, as measured using a Wyatt DynaPro Titan Dynamic Light Scattering system.

### Example 18

### Conjugation of Polysaccharide Nanoparticles with Nonenyl-Succinic Anhydride in Water

2.0 g of polysaccharide nanoparticles, produced according to Example 5, was suspended in 15 ml of water. With a pH electrode inserted into the solution, the solution was placed into a 32° C. water bath. During a 2 h period, 1.0 ml nSA was added to the solution in the following manner: the pH was constantly adjusted to 8.5 with a 4% NaOH solution and nSA was introduced into the reaction in ˜80 μl portions every 10 min. After the last portion of nSA was added to the solution, the pH kept constantly monitored and adjusted and the reaction was allowed to proceed for an additional 3 h at which point the pH was changed to 4.0 with 1M HCl.

The pellet was centrifuged (12000×g for 15 min). The pellet was re-suspended in water, the pH was adjusted to 4.0 and the solution was centrifuged in the same manner two times. Finally, the pellet was taken up in water and dialyzed against water, after the pH was adjusted to 7.0.

The mean diameter and size polydispersity of the resultant modified polysaccharide nanoparticles were 34.8 nm and 10.0% respectively, as measured using a Wyatt DynaPro Titan Dynamic Light Scattering system.

### Example 19(a)

### Cationic Polysaccharide Nanoparticles: Trimethylaminopropyl-Polysaccharide Nanoparticles

111 mg of polysaccharide nanoparticles, produced according to Example 5, was dissolved in 1.5 ml of DMSO. Then 130 mg NaOH was added and the mixture was stirred for one hour. 3 ml of (3-bromopropyl)trimethylammonium bromide solution was added to the mixture (64.66 mg/ml in DMSO), the reaction system was kept at 60° C. for 4 h, with constant stirring. After allowing the reaction system to cool to RT, 2 volumes of water (9 ml) and 28 ml of ethanol was added. The mixture was cooled to 4 CC and centrifuged at 12000×g for 15 min at 4 C. The pellet was dissolved in water (3 ml), intensively dialyzed against water and lyophilized. The degree of substitution was 3.4% as measured using NMR spectroscopy.

The mean diameter and size polydispersity of the resultant modified polysaccharide nanoparticles were 49.6 nm and 36.2% respectively, as measured using a Wyatt DynaPro Titan Dynamic Light Scattering system.

### Example 19(b)

### Cationic Polysaccharide Nanoparticles: Trimethylamino-Hydroxypropyl-Polysaccharide Nanoparticles

Polysaccharide nanoparticles, produced according to Example 5, were dissolved in DMSO at 74 mg/ml concentration. 50 μl of 4M NaOH was added to 3.2 ml of the polysaccharide nanoparticle solution and the temperature was increase to 60° C. 2.1 ml of 3-Chloro-2-hydroxypropyltrimethylammonium chloride solution (337 mg/ml concentration in water) was added in 10 portions (separated by 5 minutes) and the reaction was allowed to proceed for 24 h. After cooling the solution to room temperature, it was neutralized with HCl and the conjugated polysaccharide nanoparticles were precipitated with ethanol as described above. The degree of substitution was 7.1% as measured using NMR spectroscopy.

### Example 20

### Changing the Hydrophilic Character of Polysaccharide Nanoparticles by Methylation

Both conjugated and unconjugated polysaccharide nanoparticles were subjected to permethylation. Using dimethyl sulphoxide as the solvent, solid alkali-metal hydroxide as basic agents and methyl iodide as the methylating agent: 200 mg dry, powdered NaOH was added to 600 ul nanoPS solution (74 mg/ml in DMSO) and the mixture was stirred at RT for 10 min. After 10 min 5 ml CH₃—I solution was given to the reaction mixture and it was stirred for an additional 2.5 hrs at RT. The solution was “thinned” with 4 ml water and di-chloro-methan (DCM) was introduced to the system (4:6 volume ratio; DCM: water-DMSO). The O-methylated-nanoPS was extracted to the DCM phase with thorough mixing and the mixture was centrifuged on a clinical centrifuge for 10 min, to facilitate phase separation. The water layer was removed and replaced with clean d. water. The liquid-liquid extraction was repeated 4 more times. After the repeated extraction process the DCM phase was air dried.

The nanoPS particles appeared fully methylated as the sample was analyzed with NMR spectroscopy.

This modification resulted in water-insoluble polysaccharide nanoparticles. The material was nevertheless soluble in dichloromethane.

The effective diameter and size polydispersity of the resultant modified polysaccharide nanoparticles were 340.4 nm and 31.1% respectively, as measured in dichloromethane using a Wyatt DynaPro Titan Dynamic Light Scattering system. However, we found that methylated polysaccharide nanoparticles produce dynamic complexes with sizes ranging from 100 to 500 nm which made measurements in-consistent.

### Example 21

### Cellular Uptake of Polysaccharide Nanoparticles

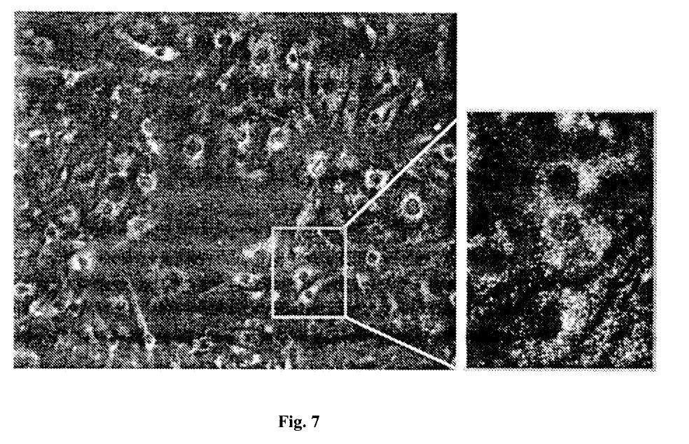

Normal murine endothelial cell lines were incubated for 16 hrs with polysaccharide nanoparticle-Rhodamine B conjugates (1.5 mg/ml), generated using the procedure described in Example 16. Fluorescence microscopy demonstrated that polysaccharide nanoparticle-Rhodamine B was taken up by normal murine endothelial cells. Polysaccharide nanoparticles were accumulated only in cytoplasmic vesicles, with no apparent surface and nucleus staining (See FIG. 7).

### Example 22

### Toxicological Assessment of Polysaccharide Nanoparticles In Vitro

The cellular toxicity of polysaccharide nanoparticles, generated according to Example 5, was compared to that of PLGA (polylactic-co-glycolic acid) nanoparticles that are commonly used in drug delivery systems. In both experiments, Hep2 cells in DMEM medium (100000 cells/ml) were incubated for 24 hrs with different concentrations of polysaccharide nanoparticles or PLGA nanoparticles. The number of dead cells as measured using the Trypan blue exclusion test and the release of LDH (lactate dehydrogenase) showed no noticeable toxicity of polysaccharide nanoparticles at a concentration of 10 mg/ml that was 2 orders of magnitude larger than concentrations shown to be toxic for PGLA nanoparticles.

### REFERENCES

1. Duncan, Nature Reviews Drug Discovery, 2 (2003) pp. 347-360
2. Manners, Carbohydrate Polymers, 16 (1991) pp. 37-82
3. Gedda et al. Bioconjugate Chem., 7 (1996) pp. 584-591
4. Holmberg et al. Bioconjugate Chem. 4 (1993) pp. 570-573
5. Lees et al. Vaccine 14 (1996) pp. 190-198
6. Bender, Eur. J. Appl. Microbiol. Biotechnol. 8 (1979) pp. 279-287
7. Coppi et al., Appl. Environ. Microbiol. 67 (2001) 3180-3187

What is claimed is:

1. A composition comprising nanoparticles comprising branched polysaccharide and wherein the nanoparticles are substantially monodisperse in size.

2. The composition according to claim 1, wherein the nanoparticles are purified from a source.

3. The composition according to claim 1, wherein the nanoparticles are further functionalized.

4. The composition according to claim 1, wherein the nanoparticles are purified from a source and further functionalized.

5. The composition according to claim 1, wherein the nanoparticles are non-toxic, biocompatible, and biodegradable.

6. The composition according to claim 1, wherein the nanoparticles are hydrophilic.

7. The composition according to claim 1, wherein the polysaccharide structure is substantially similar to that of glycogen, and is substantially free of amylopectin and amylose.

8. The composition according to claim 1, wherein the polysaccharide comprises α-D-glucose chains with 1→4 linkage and branching points occurring at 1→6 and with a degree of branching within the range of about 6% to about 13%.

9. The composition according to claim 1, wherein the polysaccharide has a polydispersity index (M_{w}/Mₙ) value between about 1.000 and about 1.100.

10. The composition according to claim 1, wherein the nanoparticles are purified from a source; the nanoparticles are non-toxic, biocompatible, and biodegradable; the nanoparticles are hydrophilic; the nanoparticles are substantially similar to glycogen; the homopolymer has a polydispersity index (M_{w}/Mₙ) value between about 1.000 and about 1.100; the nanoparticles have a spherical shape having a diameter ranging from about 20 to about 50 nm (depending on the source, and purification and isolation method); and the nanoparticles have a weight average molecular weight M_{w} ranging from about 2.00×10⁶ to about 25.00×10⁶ Da.

11. A composition comprising nanoparticles purified from a source, wherein the nanoparticles comprise at least one branched polysaccharide, and the nanoparticles are substantially spherical and substantially monodisperse in size.

12. The composition according to claim 11, wherein the source is a microorganism source.

13. The composition according to claim 11, wherein the source is a bacterial source.

14. The composition according to claim 11, wherein the source is a genetically modified bacterial source.

15. The composition of claim 11, wherein the polysaccharide has a polydispersity index (M_{w}/Mₙ) value between about 1.000 and 1.100, and wherein the nanoparticles have a diameter ranging from about 20 to about 50 nm, and wherein the polysaccharide has a weight average molecular weight ranging from about 2.00×10⁶ to about 25.00×10⁶ Da.

16. The composition according to claim 11, wherein the nanoparticles are subjected to at least one functionalization step.

17. The composition according to claim 11, wherein the nanoparticles have at least one chemical functionality attached.

18. The composition according to claim 11, wherein the nanoparticles have at least one chemical functionality attached, and wherein the chemical functionality is carbonyl, amine, hydroxyl, thiol, cyanate ester, imidocarbonate, carboxylic, or other acidic group, or unsaturated groups such as vinyl and allyl groups.

19. The composition according to claim 11, wherein the nanoparticles have at least one chemical functionality attached, and wherein the chemical functionality is attached through a homo- or hetero-bifunctional spacer or linker.

20. The composition according to claim 19, wherein the spacer or linker is diaminohexane, ethylene glycobis(sulfosuccimidylsuccinate) (sulfo-EGS), disulfosuccimidyl tartarate (sulfo-DST), dithiobis(sulfosuccimidylpropionate) (DTS SP), aminoethanethiol.

21. A composition comprising optionally functionalized nanoparticles comprising branched polysaccharide and wherein the nanoparticles are substantially monodisperse in size.

22. A composition according to claim 21, wherein the nanoparticles are purified from a source.

23. The composition of claim 21, wherein the nanoparticles are functionalized.

24. The composition of claim 21, wherein the nanoparticles are functionalized with an acidic or basic group.

25. The composition of claim 21, wherein the nanoparticles are functionalized with an electrophilic or nucleophilic group.

26. The composition according to claim 1, wherein the functionalized nanoparticles are modified by small molecule, macromolecule, biomolecule, therapeutic agents, microparticle, nanoparticle, pharmaceutically active molecule, diagnostic agent, chelating agent, dispersant, charge modifying agent, viscosity modifying agent, surfactant, coagulation agent, or flocculant.

27. The composition according to claim 21, wherein the nanoparticles are functionalized with at least one small molecule, wherein the small molecule is selected from vitamins, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics and imaging agents.

28. The composition of claim 21, wherein the nanoparticles are functionalized with at least one biomolecule, wherein the biomolecule is selected from enzymes, receptors, neurotransmitters, hormones, cytokines, cell response chemical compounds such as growth factors and chemotactic factors, antibodies, vaccines, haptens, toxins, interferons, ribozymes, anti-sense agents, and nucleic acids.

29. The composition of claim 21, wherein nanoparticles are functionalized with at least one diagnostic label, and the diagnostic label is selected from diagnostic radiopharmaceutical or radioactive isotopes, contrast agents, agents for neutron activation, and other moieties which can reflect, scatter or affect X-rays, ultrasounds, radiowaves and microwaves, and fluorophores.

30. The composition of claim 21, wherein the nanoparticles are functionalized with at least one molecule, wherein the molecule has light absorbing, light-emitting, fluorescent, Raman scattering, fluorescence resonant energy transfer, and electroluminescence properties.

31. A method of producing a polysaccharide nanoparticle, comprising:
(a) cultivating a microorganism in a growth medium in absence of phosphorous, sulfur, and/or nitrogen; and
(b) separating the biomass from the growth medium;
(c) isolating the nanoparticle from the biomass;
wherein the nanoparticle is a monodisperse, highly-branched glucose homopolymer.

32. The method of claim 31, wherein step (c) comprises:
(1) disintegrating the microorganism;
(2) obtaining a cell lyzate by separating insoluable cell components;
(3) obtaining a crude extract containing polysaccharide nanoparticles by eliminating proteins, nucleic acids, and lipopolysaccharides from the cell lyzate;
(4) purifying polysaccharide nanoparticles from the crude extract; and
(5) isolating the polysaccharide nanoparticles.

33. The method of claim 31, wherein the microorganism comprises bacteria, yeasts, microalgae, or cyanobacteria.

34. The method of claim 31, wherein the microorganism comprises bacteria.

35. The method of claim 31, wherein the microorganism is Gram-negative bacteria or rough strain Gram-negative bacteria.

36. The method of claim 31, wherein the microorganism is Geobacter sulfurreducens or rough strain Geobacter sulfurreducens.

37. The method of claim 31, wherein the microorganism is a genetically modified E. Coli.

38. The method of claim 31, wherein the microorganism is E. Coli K12.

39. The method of claim 31, wherein step (b) comprises using centrifugation or ultrafiltration.

40. The method of claim 31, wherein step (c) comprises disintegrating the microorganism which is done by French pressing or by chemical treatment.

41. The method of claim 32, wherein step (2) comprises centrifugation.

42. The method of claim 32, wherein step (3) comprises enzymatic treatment followed by dialysis.

43. The method of claim 42, wherein step (3) further comprises weak acid hydrolysis or treatment with salts of multivalent cations selected from Mg²⁺, Al³⁺, and Ca³⁺.

44. The method of claim 32, wherein step (4) comprises dialysis or size exclusion chromatography.

45. The method of claim 32, wherein step (5) comprises precipitating the polysaccharide nanoparticle with a suitable organic solvent such as acetone, methanol, ethanol, and propanol.

46. The method of claim 32, wherein step (5) comprises ultrafiltration or ultracentrifugation.

47. The method of claim 31, wherein the microorganism is bacteria selected from Gram-negative bacteria or rough strain Gram-negative bacteria.

48. The method of claim 45, wherein the bacteria is Geobacter sulfurreducens or rough strain Geobacter sulfurreducens, or E. Coli K12.

49. A method of producing a polysaccharide nanoparticle, comprising:
(a) cultivating a microorganism in a growth medium in absence of phosphorous, sulfur, and/or nitrogen; and
(b) separating the microorganism cells from the growth medium to obtain a biomass;
(c) isolating the nanoparticle from the biomass; which comprises
(1) disintegrate microorganism cells;
(2) obtaining a cell lyzate by separating insoluable cell components;
(3) obtaining an extract containing crude polysaccharides by eliminating proteins, nucleic acids, and lipopolysaccharides from the cell lyzate;
(4) purifying the crude polysaccharide nanoparticle; and
isolating the polysaccharide nanoparticle.
wherein the nanoparticle is a monodisperse, highly-branched glucose homopolymer.

50. A method of producing a polysaccharide nanoparticle, comprising:
(a) cultivating a rough strain bacteria in a growth medium in absence of phosphorous, sulfur, and/or nitrogen; and
(b) separating the bacteria cells from the growth medium to obtain a biomass using centrifugation or ultrafiltration;
(c) isolating the nanoparticle from the biomass; which comprises
(1) disintegrating the bacteria cells by French pressing or by chemical treatment;
(2) obtaining a cell lyzate by separating insoluable cell components centrifugation;
(3) obtaining an extract containing crude polysaccharides by eliminating from the cell lyzate, proteins and nucleic acids by enzymatic treatment followed by dialysis, and lipopolysaccharides by weak acid hydrolysis or treatment with salts of multivalent cations selected from Mg²⁺, Al³⁺, and Ca³⁺;
(4) purifying the crude polysaccharide nanoparticle by dialysis or size exclusion chromatography; and
(5) isolating the polysaccharide nanoparticle by precipitating the polysaccharide nanoparticle with a suitable organic solvent such as acetone, methanol, ethanol, and propanol, or ultrafiltration or ultracentrifugation.
wherein the nanoparticle is a monodisperse, highly-branched glucose homopolymer.

51. A polysaccharide nanoparticle made by the method of claim 31.

52. A polysaccharide nanoparticle made by the method of claim 49.

53. A polysaccharide nanoparticle made by the method of claim 50.

54. The polysaccharide nanoparticle of claims 51-53, wherein comprises α-D-glucose chains with 1→4 linkage and branching points occurring at 1→6 and with branching degree of about 6% to about 13%.

55. The polysaccharide nanoparticle of claim 54, wherein the nanoparticle has a polydispersity index (M_{w}/Mₙ) value between about 1.000 and 1.100.

56. The polysaccharide nanoparticle of claim 54, wherein the nanoparticle has a spherical shape having a diameter ranging from about 20 to about 60 nm.

57. The polysaccharide nanoparticle of claim 54, wherein the nanoparticle has a weight average molecular weight ranging from about 2.00×10⁶ to about 13.00×10⁶ Da.

58. A composition comprising nanoparticles comprising branched polysaccharide and wherein the nanoparticles are substantially monodisperse in size and substantially free of other polysaccharides.

59. A composition comprising nanoparticles comprising branched polysaccharide and wherein the nanoparticles are substantially monodisperse in size, and wherein the nanoparticles are substantially free of amylopectin and amylose.

60. A composition comprising nanoparticles comprising branched polysaccharide and wherein the nanoparticles are substantially monodisperse in size, wherein the composition is further substantially free of pathogenic bacteria, parasites, viruses, prions, proteins, nucleic acids, lipids and lipopolysaccharides.

61. A composition consisting essentially of nanoparticles consisting essentially of branched polysaccharide and wherein the nanoparticles are substantially monodisperse in size and substantially free of other polysaccharides.

62. A composition consisting essentially of nanoparticles comprising branched polysaccharide and wherein the nanoparticles are substantially monodisperse in size, and wherein the nanoparticles are substantially free of amylopectin and amylose.

63. A composition consisting essentially of nanoparticles comprising branched polysaccharide and wherein the nanoparticles are substantially monodisperse in size, wherein the composition is further substantially free of pathogenic bacteria, parasites, viruses, prions, proteins, nucleic acids, lipids and lipopolysaccharides.

64. A composition consisting essentially of nanoparticles consisting essentially of branched polysaccharide, and wherein proteins, nucleic acids, lipids and lipopolysaccharides cannot be detected by NMR, GC-MS, UV-VIS spectroscopy, size exclusion chromatography, fluorescence spectroscopy, XPS.

65. A method of derivatizing the polysaccharide nanoparticles of claims 51-57, comprising chemically functionalizing the nanoparticle so that its surface bears at least one chemical group such as carbonyl, amine, hydroxyl, thiol, cyanate ester, imidocarbonate, carboxylic or other acidic group, or unsaturated groups.

66. The method of claim 65, wherein the surface functional group is carbonyl, the method comprising oxidation of glucose hydroxyl groups.

67. The method of claim 65, wherein the surface functional group is amine, the method comprising reductive amination.

68. The method of claim 65, comprising cyanylation of the polysaccharide hydroxyl groups, which forms cyanate ester or iminocarbonate.

69. The method of claim 65, further comprising modifying the nanoparticles with a molecule such as by small molecule, macromolecule, biomolecule, therapeutic agents, microparticle, nanoparticle, pharmaceutically active molecule, diagnostic agent, chelating agent, dispersant, charge modifying agent, viscosity modifying agent, surfactant, coagulation agent, flocculant, or the combination thereof.

70. The method of claim 69, wherein the small molecule is selected from vitamins, anti-AIDS AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, odulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics and imaging agents.

71. The method of claim 69, wherein the biomolecule is selected from enzymes, receptors, neurotransmitters, hormones, cytokines, cell response chemical compounds such as growth factors and chemotactic factors, antibodies, vaccines, haptens, toxins, interferons, ribozymes, anti-sense agents, and nucleic acids.

72. The method of claim 69, wherein the diagnostic label is selected from diagnostic radiopharmaceutical or radioactive isotopes, contrast agents, agents for neutron activation, and other moieties which can reflect, scatter or affect X-rays, ultrasounds, radiowaves and microwaves, and fluorophores.

73. The method of claim 69, wherein the molecule has light absorbing, light-emitting, fluorescent, Raman scattering, fluorescence resonant energy transfer, and electroluminescence properties.

74. A polysaccharide nanoparticle made by the method of claims 65-69.

75. A polysaccharide nanoparticle made by the method of claim 70.

76. A polysaccharide nanoparticle made by the method of claim 71.

77. A polysaccharide nanoparticle made by the method of claim 72.

78. A polysaccharide nanoparticle made by the method of claim 73.

79. A method of using a composition for drug delivery, wherein the composition comprises optionally functionalized polysaccharide nanoparticles, wherein the nanoparticles are monodisperse, highly-branched glucose homopolymer.

80. The method of claim 79, wherein the nanoparticles comprises α-D-glucose chains with 1→4 linkage and branching points occurring at 1→6 and with branching degree of about 10%, has a polydispersity index (M_{W}/Mₙ) value between about 1.000 and 1.100, a spherical shape having a diameter ranging from about 20 to about 50 nm, and a weight average molecular weight ranging from about 2.00×10⁶ to about 25.00×10⁶ Da.

81. The method of claim 79, wherein the nanoparticles have functional group selected from carbonyl, amine, hydroxyl, thiol, cyanate ester, imidocarbonate, carboxylic or other acidic group, and is modified by the small molecule selected from vitamins, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics and imaging agents.

82. A method of using the nanoparticle of claims 51-64 for drug delivery.

83. A method of using the nanoparticle of claim 74 for drug delivery.

84. A method of using a composition for diagnosis of a disease or medical condition, wherein the composition comprising optionally functionalized polysaccharide nanoparticles, wherein the nanoparticles are monodisperse, highly-branched glucose homopolymers.

85. The method of claim 84, wherein the nanoparticles comprises α-D-glucose chains with 1→4 linkage and branching points occurring at 1→6 and with branching degree of about 10%, has a polydispersity index (M_{w}/Mₙ) value between about 1.000 and 1.100, a spherical shape having a diameter ranging from about 20 to about 70 nm, and a weight average molecular weight ranging from about 2.00×10⁶ to about 25.00×10⁶ Da.

86. The method of claim 84, wherein the nanoparticle has functional group selected from carbonyl, amine, hydroxyl, thiol, cyanate ester, imidocarbonate, carboxylic or other acidic group, and is modified by wherein the diagnostic label selected from diagnostic radiopharmaceutical or radioactive isotopes, contrast agents, agents for neutron activation, and other moieties which can reflect, scatter or affect X-rays, ultrasounds, radiowaves and microwaves, and fluorophores.

87. A method of using the nanoparticle of claim 74 for diagnosis of a disease or medical condition.

88. A method of using the nanoparticle of claim 77 for diagnosis of a disease or medical condition.

89. A method of using a composition for blood substitute product, wherein the composition comprises optionally functionalized polysaccharide nanoparticles, wherein the nanoparticles are monodisperse, highly-branched glucose homopolymer.

90. The method of claim 89, wherein the nanoparticles comprise α-D-glucose chains with 1→4 linkage and branching points occurring at 1→6 and with branching degree of about 10%, has a polydispersity index (M_{w}/Mₙ) value between about 1.000 and 1.100, a spherical shape having a diameter ranging from about 20 to about 70 nm, and a weight average molecular weight ranging from about 2.00×10⁶ to about 25.00×10⁶ Da.

91. A method of using the nanoparticle of claim 74 for blood substitute product.

92. A method of using a composition for cosmetic formulation, wherein the composition comprises optionally functionalized polysaccharide nanoparticles, wherein the nanoparticles are monodisperse, highly-branched glucose homopolymers.

93. The method of claim 92, wherein the nanoparticles comprise α-D-glucose chains with 1→4 linkage and branching points occurring at 1→6 and with branching degree of about 10%, has a polydispersity index (M_{w}/Mₙ) value between about 1.000 and 1.100, a spherical shape having a diameter ranging from about 20 to about 50 nm, and a weight average molecular weight ranging from about 2.00×10⁶ to about 25.00×10⁶ Da.

94. A method of using the nanoparticle of claims 74 for cosmetic formulation.

95. The composition according to claim 8, wherein the degree of branching ranges from about 7% to about 13%.

96. The composition according to claim 10, wherein the weight-average molecular weight ranges from about 2.00×10⁶ to about 13.00×10⁶ Da.

97. The composition according to claim 15, wherein the weight-average molecular weight ranges from about 2.00×10⁶ to about 13.00×10⁶ Da.

98. The polysaccharide nanoparticle of claim 54, wherein the degree of branching ranges from about 7% to about 13%.

99. The polysaccharide nanoparticle of claim 56, wherein the diameter ranges from about 20 to about 50 nm.

100. The method of claim 80, wherein the weight-average molecular weight ranges from about 2.00×10⁶ to about 13.00×10⁶ Da.

101. The method of claim 85, wherein the weight-average molecular weight ranges from about 2.00×10⁶ to about 13.00×10⁶ Da.

102. The method of claim 90, wherein the diameter ranges from about 20 to about 50 nm.

103. The method of claim 90, wherein the weight-average molecular weight ranges from about 2.00×10⁶ to about 13.00×10⁶ Da.

104. The method of claim 93, wherein the weight-average molecular weight ranges from about 2.00×10⁶ to about 13.00×10⁶ Da.
